(19) United States
(12) Patent Application Publication
(10) Pub. No.: US 2023/0252381 A1
(43) Pub. Date: Aug. 10, 2023

(54) ADAPTIVE VACCINE STOCKPILE

(71) Applicants: Allen BOLLANDS, Warrandyte (AU); Martina SCIONTI, Raleigh, NC (US); Dirk HOFMANN, Steinen (DE); Ethan SETTEMBRE, Lexington, MA (US); Seqirus UK Limited, Berkshire (GB)

(72) Inventors: Allen BOLLANDS, Warrandyte (AU); Martina SCIONTI, Raleigh, NC (US); Dirk HOFMANN, Steinen (DE); Ethan SETTEMBRE, Lexington, MA (US)

(21) Appl. No.: 18/002,973

(22) PCT Filed: Jun. 25, 2021
(86) PCT No.: PCT/US2021/039088

§ 371 Date: Dec. 22, 2022

Related U.S. Application Data
(60) Provisional application No. 63/144,651, filed on Feb. 2, 2021.
(60) Provisional application No. 63/045,380, filed on Jun. 29, 2020.

(51) Int. Cl. G06Q 10/0631; G06Q 10/087; G16H 40/20; G16H 50/80

(52) U.S. Cl. CPC G06Q 10/06315; G06Q 10/087; G16H 40/20; G16H 50/80

(57) ABSTRACT

A method of selectively providing access to an inventory of pre-pandemic vaccines is disclosed. The method includes identifying at least two Influenza Viruses of Pandemic Potential (IVPP) to provide available vaccines; determining a stockpile requirement for vaccines against selected IVPPs among the at least two IVPPs; obtaining finished vaccines against each of the selected IVPPs; storing an inventory of finished vaccines against each of the selected IVPPs; providing the finished vaccines against at least one of the selected IVPPs in response to an access request; and periodically obtaining further finished vaccines against one or more of the selected IVPPs and adding the further finished vaccines to the inventory of finished vaccines. Additionally disclosed are methods of selecting the inventory of finished vaccines for an adaptive vaccine stockpile.

### DESCRIPTION OF THE INVENTION

### Technical Field

The present disclosure relates to the field of supplying vaccinations, and, more particularly, methods for partially-matched vaccine supplies for use in early stages of a pandemic and to control more localized outbreaks of infection.

### Background

Many governments of the world have detailed plans which inform their response during a pandemic. Typically, central to those plans is the provision of large quantities of vaccine against the emerging pathogen. The vaccine is administered to the population, rendering them immune to infection from the pathogen, interrupting the chain of viral transmission, and bringing the pandemic to a conclusion.

However, it may take many weeks from the declaration of a pandemic until the first doses of vaccine are available. This is because of the time it takes to develop, manufacture, and test a new vaccine. During this time, the pandemic may continue to spread, hospitals may become overwhelmed and people may die. This situation is exacerbated if the emerging pathogen is entirely novel, such as COVID 19.

In order to address the lack of vaccine availability between the declaration of the pandemic and the availability of matched vaccine, some countries stockpile vaccine, usually in the form of unfinished antigen, against strains of pathogen which have a high chance of emerging as a pandemic strain, or would have a particularly high impact if they did emerge as a pandemic strain. The antigens in such stockpile are unlikely to be a precise match to any emerging pathogen. However, a stored antigen may be a close enough match to the emerging pathogen to provide a limited degree of protective immunity to recipients.

The size of such stockpiles is limited and would typically only cover a relatively small proportion of a country's population. There are a number of additional shortcomings with this traditional model of pre-pandemic vaccine stockpiling. For example, conversion of bulk antigen to finished vaccine doses requires significant time, allowing a pandemic to spread. At best, this process may take approximately eight weeks, and any other calls upon a company's manufacturing capabilities would further delay vaccine delivery. If the conversion of bulk antigen to filled and finished product is required in the early stages of a pandemic, a company's secondary manufacturing would likely be fully committed to manufacturing a fully strain-matched pandemic vaccine.

Additionally, typical antigen stored in bulk has undergone no, or very little, clinical development. In contrast, registered vaccines undergo detailed clinical development programs and are therefore highly characterized and have well-understood effects.

Further, traditional stockpile methods do not account for genetic drift in virus strains, and further, may not reflect those pathogens representing the highest risk of creating a pandemic. For example, prior to 2009, many countries were stockpiling vaccines to the H5N1 influenza virus. However, the virus which emerged as a pandemic threat in 2009 was an H1N1 strain, against which the H5N1 vaccine had zero efficacy.

Thus, there is a significant risk that the strain or strains in a stockpile will be unrelated to the emerging virus and will therefore have zero efficacy. Although this is the nature of many pathogens, such as the influenza virus, the risk is exacerbated by the fact that most pre-pandemic vaccine stockpiles are static in that the stockpiled product is not updated to track the natural mutation of viruses over time. If a virus of concern suddenly acquires the ability to transmit human-to-human, it is likely that a stockpiled vaccine that has not been periodically updated will not be the closest possible match.

Finally, stockpiled vaccines, like all vaccines, have a finite shelf life, meaning that large quantities of product must be safely disposed of upon expiration. This gives rise to a perception of wasted investment, disincentivizing an organization's decision to purchase future stockpiles.

Accordingly, a method of stockpiling vaccines is desired which can provide vaccines quickly in the early stages of a pandemic. Organizations may further benefit from stockpiles that include vaccines covering multiple different types of emerging pathogens, thereby increasing the chance that one or more vaccines may confer partial immunity on a recipient. Organizations may prefer stockpiles that remain relevant by changing to keep pace with the natural mutation of viruses, as well as remain current by maintaining sufficient supplies of non-expired vaccines to meet an organization's needs.

### SUMMARY

In one disclosed embodiment, a method of selectively providing access to an inventory of pre-pandemic vaccines is disclosed. The method includes identifying at least two Influenza Viruses of Pandemic Potential (IVPP) to provide available vaccines; determining a stockpile requirement for vaccines against selected IVPPs among the at least two IVPPs; obtaining finished vaccines against each of the selected IVPPs; storing an inventory of the finished vaccines against each of the selected IVPPs; preparing delivery logistics of the finished vaccines against at least one of the selected IVPPs in response to an access request; and periodically obtaining further finished vaccines against one or more of the selected IVPPs and adding the further finished vaccines to the inventory of finished vaccines, such that the stored inventory of finished vaccines has a sufficient number of finished vaccines that have not reached their expiration date to meet the stockpile requirement.

In an additional embodiment, methods of empirically selecting the inventory of finished vaccines for an adaptive vaccine stockpile are contemplated. In one example, the method comprises producing an initial panel of vaccines wherein the panel comprises two or more different antigens, screening combinations of two or more vaccines of the initial panel of vaccines in a non-human animal model, selecting one or more combinations for clinical testing in human patients, and selecting one or more combinations for inclusion in the inventory of finished vaccines based on the clinical testing.

### BRIEF DESCRIPTION OF THE DRAWINGS

The accompanying drawings, which are incorporated in and constitute part of this disclosure, together with the description, illustrate and serve to explain the principles of various example embodiments.

FIG. 1 is a flowchart of an exemplary method for providing access to an inventory of pre-pandemic vaccines, consistent with embodiments of the present disclosure.

FIG. 2A is a diagram of vaccine production and stockpiling, consistent with embodiments of the present disclosure.

FIG. 2B is a diagram of vaccine supply and stockpile replenishment, consistent with embodiments of the present disclosure.

FIG. 3A is a diagram of adjuvanted vaccine cross reactivity, consistent with embodiments of the present disclosure.

FIG. 3B is a diagram of cross-reactive immunity due to a panel of adjuvanted influenza vaccines, consistent with embodiments of the present disclosure.

FIG. 4 is a diagram of an exemplary development and production schedule, consistent with embodiments of the present disclosure.

FIG. 5 depicts a model of pandemic spread with and without rapid vaccine deployment, consistent with embodiments of the present disclosure.

FIG. 6 depicts a model of pandemic spread with and without rapid pre-pandemic vaccine deployment, consistent with embodiments of the present disclosure.

FIG. 7 depicts a model of a medium R0 pandemic with and without rapid pre-pandemic vaccine deployment, consistent with embodiments of the present disclosure.

FIG. 8 depicts a model of a high R0 pandemic with and without rapid pre-pandemic vaccine deployment, consistent with embodiments of the present disclosure.

FIG. 9 depicts a flow chart of an exemplary method 900 for engaging and deploying an adaptive vaccine stockpile, consistent with embodiments of the present disclosure.

FIG. 10 depicts a flow chart of an exemplary method 1000 for selecting the inventory of finished vaccines from an initial panel of vaccines, consistent with embodiments of the present disclosure.

FIG. 11 depicts an exemplary pre-clinical screening of an initial panel of four vaccines, consistent with embodiments of the present disclosure.

FIG. 12 depicts a flow chart of an exemplary vaccine deployment, consistent with embodiments of the present disclosure.

### DETAILED DESCRIPTION

Reference will now be made in detail to the example embodiments implemented according to the present disclosure, the examples of which are illustrated in the accompanying drawings. Wherever possible, the same reference numbers will be used throughout the drawings to refer to the same or like parts.

The present disclosure addresses the need of governments and other organizations to provide rapid response vaccine capability for emerging pandemics. Rapid deployment of vaccinations aid in limiting the spread of a pathogen in a population. Thus, stockpiles of developed and approved vaccinations can accelerate deployment of vaccinations by avoiding delays due to research, development and manufacturing.

However, pathogens may mutate, for example by antigenic drift, and may cause existing stockpiles to be obsolete or have reduced effectiveness. Additionally, new strains of pathogens may emerge and pose a greater pandemic threat than previous strains. For example, in 2009 a novel influenza A/California/7/2009(H1N1)pdm strain was first detected in the United States and quickly spread across the world. As reported by in Cross-Reactive Antibody Responses to the 2009 Pandemic H1N1 Influenza Virus (Hancock et al. NEJM 361, 1945-52, 2009), few young people had any existing immunity to the novel 2009 H1N1 strain but nearly one-third of people over 60 years old had at least partial immunity due to exposure to an H1N1 virus earlier in their lives. A pre-pandemic vaccine stockpiled in preparation for an emerging H5N1 virus strain, such as Aflunov® which contains antigens from an A/turkey/Turkey/1/05 (H5N1)-like strain, would offer little or no protection against the 2009 pandemic H1N1 strain.

Safeguarding against many possible pandemics is thus expensive and infeasible, particularly in the case of mutating pathogens, multiple pathogen subtypes, and changing pathogen dominance, such as for the family of flu viruses. Additional expenses are incurred due to the limited shelf life of vaccines, which force stockpiling organizations to destroy stored but unused vaccines, discouraging stockpile renewal. Accordingly, embodiments of the present disclosure provide methods to selectively provide access to an inventory of pre-pandemic vaccines, such as vaccines for influenza viruses. Vaccines for other types of pathogens, such as prions, bacteria, and viruses, are envisioned, including, but not limited to, vaccines for anthrax, plague, smallpox, measles, arenaviruses, bunyaviruses, flaviviruses, coronaviruses, ebola viruses, zika viruses and filoviruses. Vaccines for pathogens affecting animals are also envisioned.

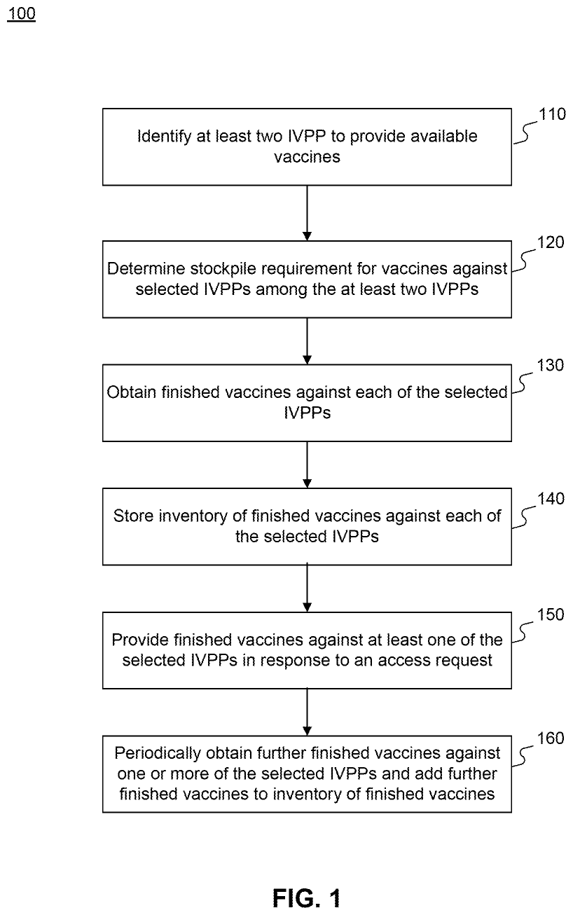

FIG. 1 is a flowchart of an exemplary method 100 for providing access to an inventory of pre-pandemic vaccines, consistent with embodiments of the present disclosure. Method 100 may be performed, for instance, by a vaccine supplier.

Step 110 includes identifying at least two Influenza Viruses of Pandemic Potential (IVPPs) to provide available vaccines. A vaccine manufacturer may consult subject matter experts and study emerging influenza viruses to determine which strains or subtypes are most likely to cause a pandemic, or which would cause the most impact if they did cause a pandemic. For instance, identified IVPPs may include at least one IVPP chosen from H1, H2, H3, H5, H7, or H9 subtypes. Other subtypes may be considered as well, including any subtype of influenza A, B, C, or D, or any other subtypes that may be identified in the future. IVPPs may also be identified according to neuraminidase proteins, such as N1 or N5. Specific strains of influenza virus subtypes may also be identified, such as H3N2 strain A/Kansas/14/2017.

The IVPPs may be identified with reference to an Influenza Risk Assessment Tool (IRAT). Experts may use an IRAT to combine measurements of the emergence risk of an influenza virus, including the risk that a new virus will acquire the ability to spread among a population, and the impact of an influenza virus, such as the severity of illness and burden on society due to those who are infected. Additionally, experts may consider properties of the virus, such as transmission in animal models, attributes of the population, such as existing population immunity, and ecology and epidemiology, such as necessary conditions for the virus to infect humans. Identification may be performed in conjunction with governmental health organizations, such as the Centers for Disease Control and Prevention (CDC), WHO, or state-level health agencies. Scores may be quantitative or qualitative based on expert judgement. IVPPs may then be identified based on both the emergence and impact scores obtained with the IRAT.

Although the impact to the entire population may be low for a particular strain, the impact may be high for subsets of the population, and IVPPs may be determined from IRATs focusing on population subsets. In some embodiments, an access request and/or a stockpile requirement may be identified based on at risk groups comprising, for example young children, the elderly, individuals with co-morbidities, first responders, and other essential workers. For instance, a given country may have a population with large numbers of children. If an influenza strain is identified as having a dangerous impact on children, but not adults and elderly citizens, IVPPs may be determined from IRATs focusing on the impact to children, rather than the population as a whole. Additionally, if a portion of a population already has some immunity to an influenza strain, IVPPs may be determined from IRATs focusing on the portion of a population that does not have immunity. For example, if a strain of influenza was responsible for a pandemic thirty years ago, people over the age of thirty may have some immunity due to previous exposure, but people under thirty may have no immunity. Further, IVPPs may be determined to protect first responders or other essential workers, such as paramedics, doctors, nurses, police, firefighters, military, food distribution workers and utility workers. For example, a particular influenza strain that affects working-age people after repeated contact may have a greater impact on doctors and nurses than on the remainder of the population, and the particular influenza strain may be identified as an IVPP so that a government or organization may protect first responders.

Step 110 may also include receiving information regarding a desired number of doses of vaccines and a selection of at least one IVPP from the identified at least two IVPPs. A government may provide information regarding a desired number of doses of vaccines for its citizens, based, for instance, on population sizes of specific target cohorts such as elderly, young, working people, people with comorbidities (including those who are immunocompromised, or have COPD, asthma, or high blood pressure). Additionally, the information may be received from a governmental organization, such as a department of health, emergency response organizations, or military, thus providing coverage for first responders and essential workers. Non-governmental organizations, such as charity services or emergency aid organizations, businesses, and medical providers may also provide information regarding a desired number of doses of vaccines. In some embodiments, such as for vaccines for animals, agricultural organizations may provide information to protect livestock including species, breeds, farming practices, and past infections. Further, the desired number of doses may be based on epidemiological modeling, such as models that determine the portion of a target population that should be immunized to contain an outbreak.

Step 120 includes determining a stockpile requirement for vaccines against selected IVPPs among the at least two IVPPs. The stockpile requirement may be determined based on received information regarding one or more factors comprising a desired number of doses of vaccines and a number of selected IVPPs. Further, a subscription fee for access to the stockpile may be an annual fee calculated based on the stockpile requirement. For example, in some embodiments, delivery may be promised no more than two weeks following an access request. However, if a government requests that the delivery occur in a shorter period of time, such as within three days of a request, the subscription fee may increase in order to retain sufficient delivery resources to meet the request, including air transportation contracts, on-demand trucking contracts, or purchase of trucks and hiring drivers to ship doses on short notice. Establishing storage sites near delivery sites to reduce shipping time may also be required, and increase an annual fee. Additional doses and multiple selected IVPPs require additional storage space, which may also increase cost. Additionally, each IVPP may require research, development and manufacturing startup costs, causing a greater annual fee if an organization desires vaccines against multiple IVPPs. In some embodiments, the type of dose delivery may also affect the subscription fee. For example, pre-filled syringes may be more expensive to manufacture and store than multi-dose vials. However, pre-filled syringes may be more convenient for a hospital to administer in an emergency situation. Therefore, a hospital may prefer paying a higher subscription fee in exchange for access to pre-filled syringes instead of multi-dose vials. Delivery location may also affect the subscription fee. For instance, a country may have stringent import regulations, increasing logistics costs, or lengthy vaccine approval and regulatory processes, increasing research and legal costs. Thus, the country may be charged an increased annual fee for access.

Step 130 includes obtaining finished vaccines against each of the selected IVPPs. If a vendor performs steps 110 through 120, the vendor may purchase vaccines from a vaccine manufacturer at step 130. Alternatively, if a manufacturer performs steps 110 through 120, the manufacturer may produce vaccines at step 130. Finished vaccines that contain antigens, stabilizers, adjuvants, antibiotics, preservatives, and the like may be obtained. In some embodiments, bulk antigen may be obtained, rather than or in addition to finished vaccines. This may reduce storage cost, but also increase the length of time needed to deliver vaccines due to the time needed to finish the vaccines. Step 130 may include research and development of a vaccine against an IVPP, testing, regulatory approval and other ancillary tasks. Additionally, bulk antigen may be stored in order to quickly restock stockpiles of finished vaccines or provide a quicker ramp-up of production of finished vaccines.

Step 140 includes storing an inventory of finished vaccines against each of the selected IVPPs. A manufacturer may maintain the inventory of target vaccines, for instance, near the manufacturing site. If bulk antigen, rather than finished vaccines, are maintained, storage near the manufacturing site may enable quicker finishing and delivery of vaccines during an outbreak. Alternatively or additionally, a vendor may maintain the inventory of target vaccines. A vendor may have flexibility in storage location. For instance, a vendor may build or contract for a warehouse near a customer's desired delivery site, reducing shipping delays.

Further, finished vaccines may be stored as multi-dose vials, pre-filled syringes, freeze-dried vaccines, vaccine patches, nasal vaccines, or any combination thereof. A customer may prefer a mix of more expensive pre-filled syringes and less expensive multi-dose vials, depending on customer needs. Additionally, multi-dose vials may have longer shelf-lives, requiring less frequent manufacturing, but also increasing the risk of obsolescence due to antigenic drift over the shelf life of the vaccine. Therefore, a combination of pre-filled syringes and multi-dose vials may provide a hedge against antigenic drift while reducing customer's annual fee.

Step 150 includes preparing delivery logistics of the finished vaccines against at least one of the selected IVPPs in response to an access request. A customer may request the finished vaccines when a certain threshold of infections is reached, for instance. A vendor or manufacturer may place requirements on delivery, such as a minimum quantity. In case of a pandemic, a customer may prioritize distribution of doses among an affected population. Priority may be given to medical workers or first responders, or to locations where a virus has already become widespread. Alternatively, if one region has widespread infection, such that late vaccinations will not have a substantial effect in stopping the spread, while another region has isolated cases, the region with isolated cases may be prioritized to contain an outbreak. Further, a region with a large population of vulnerable groups, such as children, may receive vaccinations prior to a region with invulnerable groups, such as elderly who have already achieved immunity to the virus strain due to past exposure. In some embodiments, a customer may only receive the finished vaccines after the finished vaccines are drawn down following an access request.

Step 160 includes periodically obtaining further finished vaccines against one or more of the selected IVPPs and adding the further finished vaccines to the inventory of finished vaccines, such that the stored inventory of finished vaccines has a sufficient number of finished vaccines that have not reached their expiration date to meet the stockpile requirement. Step 160 may address the need to refresh a stockpile due to expiration of stored vaccines. For example, finished vaccines may have a shelf life of at least 2 years. In this case, vaccine stockpiles may need to be replaced with further finished vaccines prior to 2 years, or more frequently, to ensure an in-date vaccine is available whenever a customer may request delivery. In this way, finished vaccines may be replaced in the inventory prior to the expiration date.

Additionally, in some embodiments, the inventory of finished vaccines may be updated to account for antigenic drift. For example, if expert advisers recommend that a stockpiled vaccine against a specific strain of virus be updated because of the antigenic drift of the target virus strain, a vendor or manufacturer may replace expiring vaccines designed to protect against the earlier strain with a vaccine against the later, drifted but more prominent or impactful, strain. Additionally or alternatively, identified IVPPs may be updated. For instance, new IVPPs may be identified prior to replacement of an expiring inventory of vaccines by using an IRAT with updated epidemiological information. This may reveal that, although H3 and H7 were IVPPs when an expiring vaccine stock was originally manufactured, a strain of H5 has since emerged with a greater impact than H3, thus requiring a new inventory of H5 to replace the expiring inventory of H3.

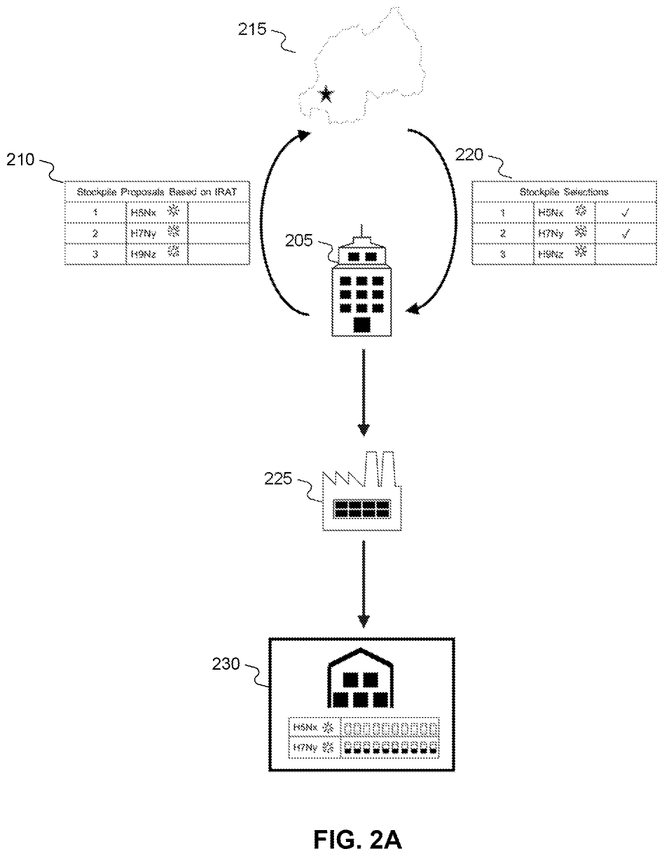

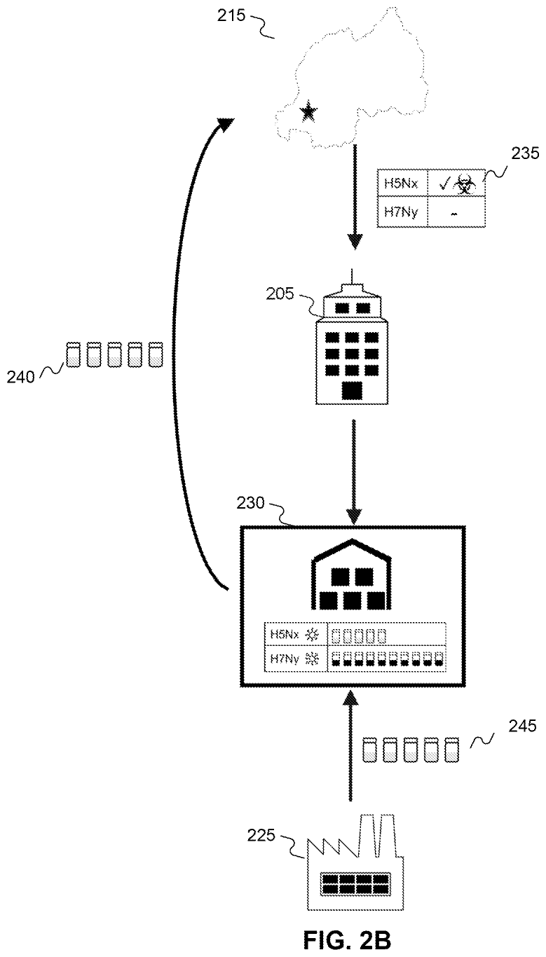

FIG. 2A is a diagram of vaccine production and stockpiling, consistent with embodiments of the present disclosure. In FIG. 2A, vaccine vendor 205 provides stockpile proposals based on IRAT 210 including a list of IVPPs to a country 215. The stockpile proposals 210 may also include additional information beyond the information considered in an IRAT, threat assessments, demographic impacts, and the like. As illustrated in FIG. 2A, vendor 205 may identify H5, H7, and H9 as IVPPs. Country 215 then provides vendor 205 with stockpile selection 220 of at least one IVPP. The selection may be based on economic considerations. For instance, country 215 may have a budget for vaccines for only two IVPPs, and select H5 and H7, as illustrated in stockpile selection 220. Vendor 205 then instructs manufacturer 225 to produce a number of doses based on the country's needs for each of the selected IVPPs, and stores the doses in a warehouse 230. Additionally, vendor 205 periodically provides country 215 with new stockpile proposals 210, updated to reflect the most current threats, as well as an opportunity to add or change stockpile composition consistent with the strains being offered in stockpile proposals 210. In some embodiments, vendor 205 may alternatively or additionally provide status updates advising a customer that a new vaccine has replaced a stockpiled vaccine due to antigenic drift. For example, a country may request a stockpile of H5N1 vaccines. A first batch of vaccine may be based on a certain strain of H5N1, and the manufacturer may inform the country when a new vaccine for a different strain of H5N1 has replaced an older vaccine in the stockpile. In some embodiments, any combination of vendor 205, manufacturer 225, and warehouse 230 may be co-located. For instance, manufacturer 225 may include an on-site storage facility functioning as warehouse 230. Further, any combination of vendor 205, manufacturer 225, and warehouse 230 may be co-owned and/or operated.

FIG. 2B is a diagram of vaccine supply and stockpile replenishment, consistent with embodiments of the present disclosure. Country 215 may determine that an H5 influenza virus is spreading among its population. Country 215 sends an access request 235 to vendor 205 for doses of H5 vaccine 240. The number of doses of H5 able to be ordered is no more than the number of doses of H5 vaccine stored in warehouse 230 on behalf of country 215. Doses of H5 vaccine 235 are delivered from warehouse 230 to country 215. As the inventory of H5 doses in warehouse 230 is drawn down, manufacturer 225 produces replacement doses 245 to replenish the inventory in warehouse 230 at the earnest practical opportunity. Replacement doses 245 may be created after manufacturer redirects its processes to produce new original doses of H5 vaccine. Alternatively, manufacturer 225 may convert bulk antigen into finished doses to more quickly resupply warehouse 230. Conversion of bulk antigen may also avoid additional regulatory and approval tasks, further accelerating delivery to warehouse 230. In some cases, manufacturer 225 may deliver replacement doses 245 directly to country 215, such as when a virus spreads quickly and country 215 orders additional doses beyond the number of doses in the stockpile of country 215.

As previously stated, vaccine stockpiles may enable a fast response to an emerging outbreak, slowing spread of the pandemic or helping contain an outbreak, thereby providing additional time for an organized societal and scientific response. Additionally, providing vaccines that protect against a range of influenza viruses, rather than being limited to a particular strain, increases the likelihood that one of the stockpiled vaccines will be effective, thereby improving the expected benefit of stockpiling vaccines, encouraging investment in prophylactic measures rather than relying on remedial actions, and helping to diminish the impact of pandemics.

Accordingly, in some embodiments of the present disclosure, a vaccine manufacturer may produce a vaccine that leads to the creation of antibodies capable of targeting a plurality of pathogens. In other words, a manufacturer producing influenza vaccines may produce a vaccine that leads to cross reactive immunity for a number of viruses, such as those having a common clade, rather than a single strain, Cross-reactivity may occur naturally, such that a vaccine for H5N1 produces some immunity for H5N6. Thus, in some scenarios, a country may deploy (e.g., draw down and deliver to a designated drop-off point) a pre-pandemic vaccine even though a pandemic virus is from a different strain so that cross-reactivity may help slow the pandemic, even if the cross reactive effect is small.

Further, cross-reactive immunity may occur due to the presence of an adjuvant, such as MF59, an oil-in-water emulsion containing squalene which has been shown to induce cross reactive immunity for influenza viruses. For example, AFLUNOV® is an influenza vaccine containing MF59 and antigens of a A/turkey/Turkey/1/05 (H5N1)-like strain. AFLUNOV® produces immunity in humans for A/turkey/Turkey/1/05, and also elicits cross reactive immunity against a range of H5 clades that have caused human disease, such as A/Mongolia/244/05.

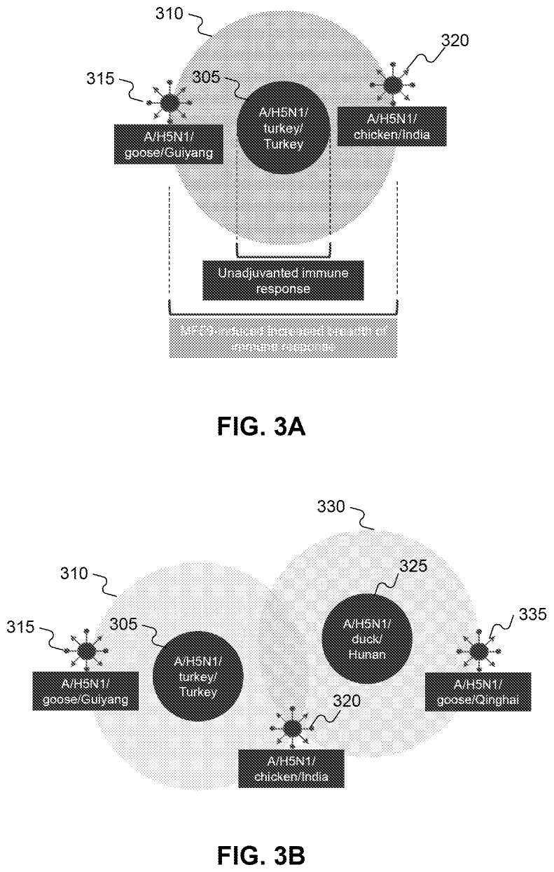

Cross reactivity may be further understood by reference to FIG. 3A showing a diagram of vaccine cross reactivity, consistent with embodiments of the present disclosure. FIG. 3A illustrates the reactivity of an unadjuvanted influenza vaccine in comparison to the cross reactivity of an adjuvanted influenza vaccine. For example, an unadjuvanted H5N1/turkey/turkey strain-based vaccine induces an immune response 305 which protects against the H5N1/turkey/Turkey strain. However, when the same vaccine is administered with an adjuvant such as MF59, the vaccine may also induce the production of antibodies which neutralize non-H5N1/turkey viruses, and leads to at least a partial immunity to other influenza strains, For example, as shown in FIG. 3A, adjuvanted H5N1/turkey/Turkey strain immune response 310 may provide at least partial immunity to H5N1/goose/Guiyang strain viruses 315 and H5N1/chicken/India strain viruses 320, In other words, an adjuvanted vaccine against the H5N1/turkey/Turkey strain may provide some protection against the H5N1/goose/Guiyang strain and H5N1/chicken/India strain. In some embodiments, vaccines may be stored and used with no adjuvant. In some embodiments, a vaccine may be delivered to a customer along with a supply of adjuvant to help improve cross reactivity. For example, a stockpiled vaccine may have some cross-reactivity to cover an emerging pandemic strain, but, when adjuvanted, provides greater individual or populational immunity. Thus, a customer may request delivery of adjuvant that may be mixed with the vaccine by medical professional, such as near the time of administering the vaccine.

In some embodiments, the adjuvanted immune response may not induce total immunity in a vaccinated population for an additional strain, but rather partial immunity in a vaccinated population. For example, some people may gain immunity to a plurality of strains from a single course of a specific vaccine, while others only gain immunity to the targeted strain. Additionally, the adjuvanted vaccine may produce a partial, rather than total, immunity in individuals, such that a person receiving an influenza vaccine based on the H5N1/turkey/Turkey strain may still become ill due to the A/H5N1/goose/Guiyang strain, but may also recover quicker than an unvaccinated person.

FIG. 3B is a diagram of cross-reactive immunity due to a panel of adjuvanted influenza vaccines, which when stockpiled may provide broader coverage against emergent and/or drifted antigens than just the specific target antigens, consistent with embodiments of the present disclosure. For example, a vaccine may include antigens for both the A/H5N1/turkey/Turkey strain and the A/H5N1/duck/Hunan strain. If the vaccine is unadjuvanted, the vaccine may not provide protection against other strains. For example, unadjuvanted H5N1/turkey/Turkey strain immune response 305 and unadjuvanted H5N1/duck/Hunan strain immune response 325 do not provide immunity to H5N1/goose/Guiyang strain viruses 315, H5N1/chicken/India strain viruses 320, or H5N1/goose/Qinghai strain viruses 335, Thus, in order to gain immunity from all five strains, five vaccinations would be needed. Immunizing a population against all five strains may be impossible due to cost or dangerous medical interactions.

However, adjuvanted H5N1/turkey/Turkey strain immune response 310 and adjuvanted H5N1/duck/Hunan strain immune response 330 together provide broader protection than the unadjuvanted variations. Further, a partial immunity achievable by a single adjuvanted vaccine may be broadened by addition of a second adjuvanted vaccine. For example, adjuvanted H5N1/turkey/Turkey strain immune response 310 and adjuvanted H5N1/duck/Hunan strain immune response 330 may provide some immunity to H5N1/chicken/India strain viruses 320. This may occur, for instance, if A/H5N1/chicken/India strain viruses have surface proteins similar to either A/H5N1/turkey/Turkey or A/H5N1/duck/Hunan viruses, and the greater number of antibodies created in response to both antigens increases the probability of an immune response to the H5N1/chicken/India strain as well. Thus, either one of the stockpiled H5N1/turkey/Turkey or H5N1/duck/Hunan pre-pandemic vaccines may be administered in adjuvanted form in response to a pre-pandemic H5/N1/chicken/India virus. In certain embodiments, the adjuvanted H5N1/turkey/Turkey pre-pandemic vaccine may be administered to one portion of a population and the adjuvanted H5N1/duck/Hunan pre-pandemic vaccine administered to another portion of the population. In other embodiments, the adjuvanted H5N1/turkey/turkey and H5N1/duck/Hunan pre-pandemic vaccines may be co-administered to individuals in a strategy of Prime-Boost to provide broader protection to the A/H5/N1/chicken/India virus. Thus, it may be possible for two stockpiled pre-pandemic vaccines (e.g., H5/N1/turkey/Turkey and H5/N1/duck/Hunan) to produce at least a partial immunity for multiple different virus strains. In this way, a stockpile of adjuvanted vaccine may provide protection against more than two strains of a virus.

In some embodiments, a stockpile may include vaccines that are bivalent, trivalent, or any other plurality. A bivalent or trivalent vaccine may include a variety of different antigens (e.g., H5N1 and H1N1), a variety of different Glades of the same antigen (e.g., multiple clades of H5N1) or a combination thereof. The combined effect of such multi-valent options may encourage organizations to obtain a pre-pandemic vaccine stockpile, because they may have protection against more pandemic scenarios than achievable with unadjuvanted or monovalent vaccines.

Due to the limited shelf-life of manufactured vaccines, production and storage of influenza vaccine inventories may be determined according to expiration dates. For example, as stated previously, step 170 of process 100 includes periodically obtaining further finished vaccines against one or more of the selected IVPPs and adding the further finished vaccines to the inventory of finished vaccines. The period of step 170 may be based on shelf life of an inventory.

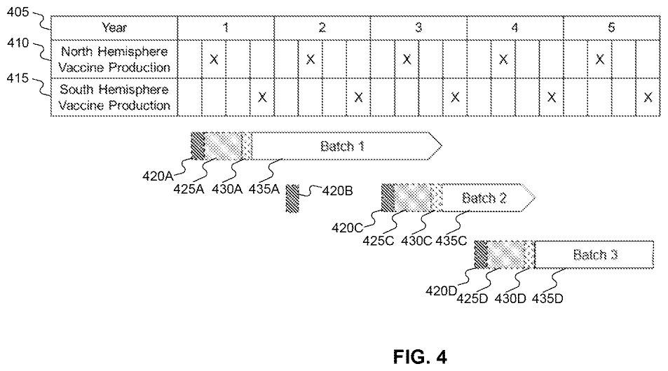

FIG. 4 is a diagram of an exemplary development and production schedule, consistent with embodiments of the present disclosure. The schedule shown in FIG. 4 presents a notional planning and production schedule, and is not intended to be limiting. Multiple permutations of the schedule presented in FIG. 4 are envisioned, which, for instance, begin at different periods of a year, last for different durations, or occur in different sequences from the illustration.

In FIG. 4, northern hemisphere vaccine production 410 and southern hemisphere vaccine production 415 are aligned to years 405. For example, the northern hemisphere vaccine production may occur in the second quarter of each year, and the southern hemisphere vaccine production may occur in the fourth quarter of each year, aligned to the timing of seasonal influenza in each hemisphere. Thus, a production window between the two vaccine production periods may exist to accommodate manufacture of pre-pandemic vaccines for a stockpile.

Additionally, other planning and coordination may be scheduled in order to ensure that manufacturing occurs during these annual windows. In some cases, virus selection, research, development, procurement, and regulatory approvals may precede vaccine production.

For example, as illustrated in FIG. 4, storage of batches 1, 2, and 3 are preceded by IVPP selection periods 420A, 420B, 420C, and 420D; preparatory phases 425A, 425C, and 425D; and manufacturing phases 430A, 430C, and 430D. Each batch also has a storage phase 435A, 435C, and 435D. In some embodiments, approximately six months of planning may precede storage of vaccine stockpile.

Batch 1 of FIG. 4 is preceded by IVPP selection period 420A, occurring during the first quarter of year 1. During this time, experts identify an IVPP and recommend production of a new vaccine. Preparatory phase 425A then begins during the second quarter of year 1, so that a manufacturer or vendor may perform research, develop a vaccine, and acquire materials and regulatory approval. Preparatory phases may be longer, for instance, in the case of a novel vaccine, or shorter, such as for a vaccine that is a dose variant of an already-approved vaccine. Variations in preparatory phase length may alter the timing of subsequent phases. Manufacturing phase 430A ends in the third quarter of year 1, prior to the southern hemisphere vaccine period, and batch 1 enters storage 435A until deployment or expiration approximately two years later.

An annual IVPP selection period 420B occurs during year 2. In the scenario presented, experts determine that batch 1 in storage 435A provides protection against selected IVPPs, and that no new IVPPs have arisen. Thus, no preparatory or manufacturing phases occur.

During year 3, experts at an annual IVPP selection period 420C identify a new IVPP, and determine that a new vaccine is necessary. Alternatively, experts may decide that, because batch 1 expires during year 3, a new batch must be produced to maintain a current stockpile of vaccines. In either scenario, preparatory phase 425C and manufacturing phase 430C follow IVPP selection period 420C. Batch 2 then enters storage 435C as batch 1 expires during the fourth quarter of year 3.

IVPP selection period 420D occurs in year 4. In this case, experts identify a new IVPP which is not covered by the vaccine of batch 2. Preparatory phase 425D and manufacturing phase 430D create batch 3, which enters storage 435D at the beginning of the fourth quarter of year 4. Once batch 3 is manufactured, batch 2 may be destroyed, as illustrated by the shorter duration of batch 2 storage 435C. Alternatively, batch 2 may remain in storage, as its shelf life of two years has not yet expired.

Thus, as illustrated, IVPP selection periods may be determined by the manufacturing windows between annual seasonal vaccine production cycles, as well as the duration of any planning and preparatory work. Further, batches may be replaced due to expiration or obsolescence, and planning and preparatory work may be omitted if a batch remains relevant.

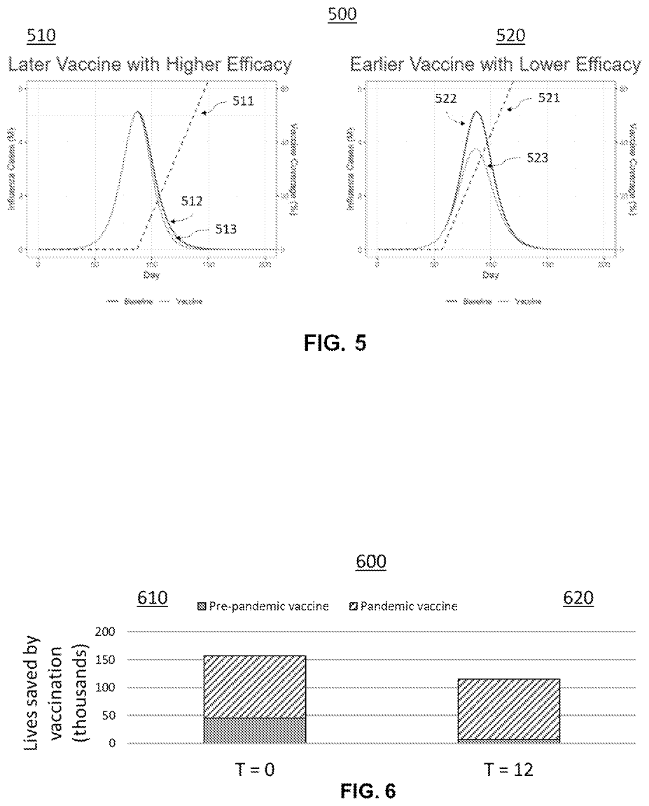

Further embodiments of the invention contemplate additional methods of producing the inventory of vaccines. In the early stages of a pandemic, rapid deployment of an imperfectly matched less-efficacious vaccine may be more beneficial than a slowly deployed but better matched highly-efficacious vaccine. Typically, availability of a first matched pandemic vaccine using current technologies may be at least 16 weeks. However, morbidity and/or mortality of first responders and essential workers in the early days of a pandemic may critically impair a pandemic response strategy. Accordingly, a partially-matched vaccine administered early may deliver valuable public health benefits. However, many bulk antigen pre-pandemic stockpiles have several shortcomings such as (i) slow deployment of bulk antigen which typically takes a minimum of 8 weeks and often 10-12+ weeks to be converted to usable product, (ii) minimal clinical data as the vaccine may be uncharacterized and unregistered, and (iii) a finite shelf-life as vaccines eventually expire and must be written off. By contrast, an adaptive vaccine stockpile may rapidly deploy finished and registered vaccines. Exemplary benefits of a rapidly deployable adaptive vaccine stockpile are illustrated in FIGS. 5-8.

FIG. 5 depicts a model comparing influenza spread with later deployment of a highly efficacious vaccine 510 versus influenza spread with rapid deployment of a less efficacious vaccine 520. Dotted lines 511 and 521 depicts vaccine coverage with time, lines 512 and 522 depict influenza cases with time without vaccination, and lines 513 and 523 depict influenza cases with vaccination. In particular, comparing 510 to 520 demonstrates earlier administration of a less efficacious vaccine may result in fewer influenza cases. Accordingly, FIG. 5 demonstrates benefits associated with a rapid vaccine deployment.

FIG. 6 depicts Monte Carlo simulations modeling the impact of rapid vaccine deployment. FIG. 6 side 610 depicts a rapid deployment of a less efficacious pre-pandemic vaccine from T=0 followed by later deployment of a more efficacious pandemic vaccine from T=16 weeks. FIG. 6 side 620 depicts a 12 week delayed deployment of a less efficacious pre-pandemic vaccine caused by, for example, conversion of bulk antigen to finished product, followed by deployment of a more efficacious pandemic vaccine from T=16 weeks. FIG. 6 demonstrates that early availability of a pre-pandemic vaccine may save lives. For example, in FIG. 6 side 610, a pre-pandemic vaccine administered from T=0 saves 46,000 lives, approximately 29% of total lives saved by vaccination, and the matched pandemic vaccine administered from T=16 saves an additional 111,000 lives. By contrast, in FIG. 6 side 620, a pre-pandemic vaccine administered from T=12 saves 7,000 lives, approximately 6% of total lives saved by vaccination, and the matched pandemic vaccine saves a further 108,000 lives. In FIG. 6, T=0 denotes the time of pandemic declaration and T=12 denotes 12 weeks after pandemic declaration. Simulations were run using (i) a range of basic reproduction values, R0, from 1.1 to 1.8, (ii) administration of 20 million doses of a pre-pandemic vaccine having an efficacy of 17%, and (iii) administration of a matched pandemic vaccine at 16 weeks.

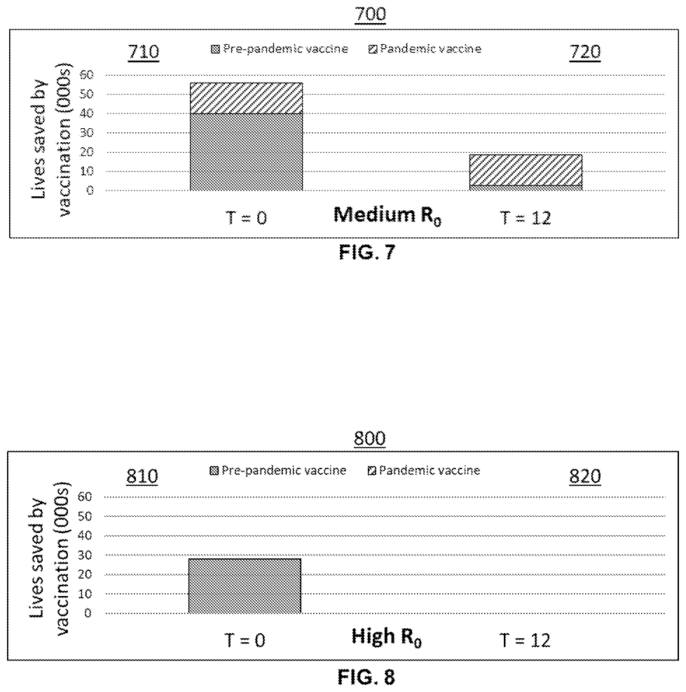

FIGS. 7 and 8 depict the increasing importance of rapid vaccine deployment with increasing R0. FIG. 7 side 710 depicts a Monte Carlo simulation of a medium R0 pandemic with a partially matched (17% effective) pre-pandemic vaccine deployed from T=0 to 20 million people followed by a matched pandemic vaccine deployed from 16 weeks. By comparison, FIG. 7 side 720 depicts deployment of the pre-pandemic vaccine from T=12 followed by a matched pandemic vaccine from 16 weeks.

FIG. 8 side 810 depicts a Monte Carlo simulation of a high R0 pandemic with a partially matched (17% effective) pre-pandemic vaccine deployed from T=0 to 20 million people followed by a matched pandemic vaccine from 16 weeks. By comparison, FIG. 8 side 820 depicts deployment of the pre-pandemic vaccine from T=12 followed by a matched pandemic vaccine from 16 weeks. A high R0 virus may sweep through a population before a matched pandemic vaccine is available. As such, the matched pandemic vaccine may have negligible benefit. Greater benefits can accrue for a partially-matched pre-pandemic vaccine if deployed from, for example, T=4 as may happen in an emerging pandemic.

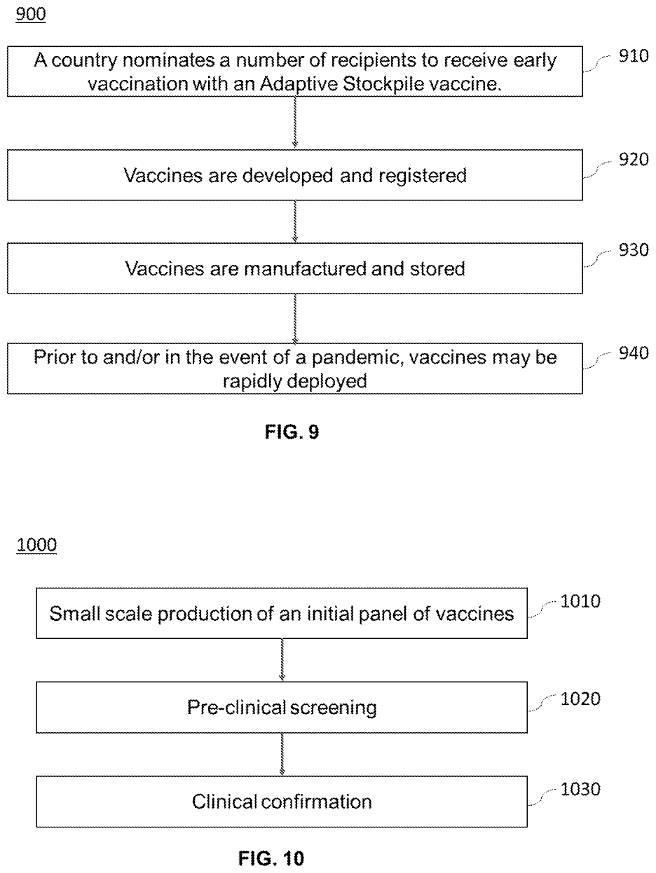

FIG. 9 depicts a flow chart of an exemplary method 900 for engaging and deploying an adaptive vaccine stockpile consistent with embodiments of the present disclosure. In step 910, a country may nominate a number of recipients to receive early vaccination with an adaptive stockpile vaccine. Step 910 may be performed before and/or independently of the exemplary method 900. In step 920, vaccines may be developed and registered for inclusion in an adaptive vaccine stockpile. Vaccine development may comprise, for example, small scale production, pre-clinical testing, and/or clinical testing of an initial panel of vaccines for inclusion in an adaptive vaccine stockpile. Registration of a vaccine may comprise registering a vaccine with a regulatory body such as, for example, the United States Food and Drug Administration. In step 930, vaccines are manufactured and stored. Vaccines may be manufactured and stored in filled and finished form to facilitate rapid deployment. An exemplary embodiment comprises a panel of monovalent cell-based MF59-adjuvanted pre-pandemic vaccines in a final container format. In step 940, vaccines may be rapidly deployed prior to and/or in the event of a pandemic. Any of steps 920-940 may be performed using a vendor-maintained subscription model. Steps 920 and 930 may be independently iterated to update the adaptive vaccine stockpile to account for, for example, IVPP drift and/or vaccine expiration.

As previously stated, organizations may further benefit from stockpiles that include vaccines covering multiple different types of emerging pathogens, thereby increasing the chance that one or more vaccines may confer partial immunity on a recipient. Accordingly, the inventory of finished vaccines may be selected to increase the coverage of the stockpile.

FIG. 10 depicts a flow chart of an exemplary method 1000 for selecting the inventory of finished vaccines from an initial panel of vaccines consistent with embodiments of the present disclosure.

Step 1010 includes small scale production of an initial panel of vaccines. Vaccines may be adjuvanted with MF59 to increase heterologous coverage. The initial panel of vaccines may comprise two or more different antigens. For example, the initial panel of vaccines may comprise two or more different influenza strains. The initial panel of vaccines may be selected for antigenic diversity. For example, the initial panel of vaccines may be selected to increase the number of different epitopes present in the initial panel of vaccines. The initial panel of vaccines may be produced at a small scale in accordance with good manufacturing practice (GMP) procedures. Small scale manufacturing may be scaled up as needed.

Step 1020 includes pre-clinical screening of the initial panel of vaccines. Pre-clinical screening may comprise characterizing, for two or more vaccines in the initial panel, cross-reactivity against different influenza strains, reactivity against recent influenza strains, and/or reactivity against different influenza subclades. Pre-clinical screening may be performed using a non-human animal model such as, for example, ferrets.

FIG. 11 depicts an exemplary pre-clinical screening 1100 of an initial panel of four vaccines 1110 consistent with embodiments of the present disclosure. The four vaccines in the exemplary initial panel 1110 are characterized in pair-wise combinations, e.g. 1120, for reactivity to and/or coverage of seven different viral strains 1130. The protection conferred by each pair-wise combination may be characterized, and the best pair-wise combination(s), e.g. 1140, may be selected for clinical confirmation. Determining the best combinations of vaccines may comprise determining a combination of vaccines' response against multiple strains, determining a combination of vaccines' response against recent strains, and/or determining a combination of vaccines' response against disparate subclades.

Step 1030 of FIG. 10 includes clinical confirmation in human patients of the pre-clinical screening results. Clinical confirmation may comprise characterizing, in human patients, two or more vaccines in the initial panel for cross-reactivity against different influenza strains, reactivity against recent influenza strains, and/or reactivity against different influenza subclades. Clinical confirmation may comprise characterizing, in human patients, two or more vaccines in the initial panel for safety and efficacy.

In some embodiments, a method of empirically selecting the inventory of finished vaccines comprises producing an initial panel of vaccines wherein the panel comprises two or more different antigens, screening combinations of two or more vaccines of the initial panel of vaccines in a non-human animal model, selecting one or more combinations for clinical testing in human patients, and selecting one or more combinations for inclusion in the inventory of finished vaccines based on the clinical testing. In some embodiments, the initial panel of vaccines may be updated to account for antigenic drift. In some embodiments, the initial panel of vaccines may be selected with reference to an IRAT. In some embodiments, the initial panel of vaccines may be selected using antigenic cartography.

As previously stated, rapid deployment of vaccinations aid in limiting the spread of a pathogen in a population. Accordingly, in some embodiments, the inventory of finished vaccines is selected with reference to an IRAT for a rapid initial response, and the inventory of finished vaccines is updated one or more times using the method of empirically selecting the inventory of finished vaccines.

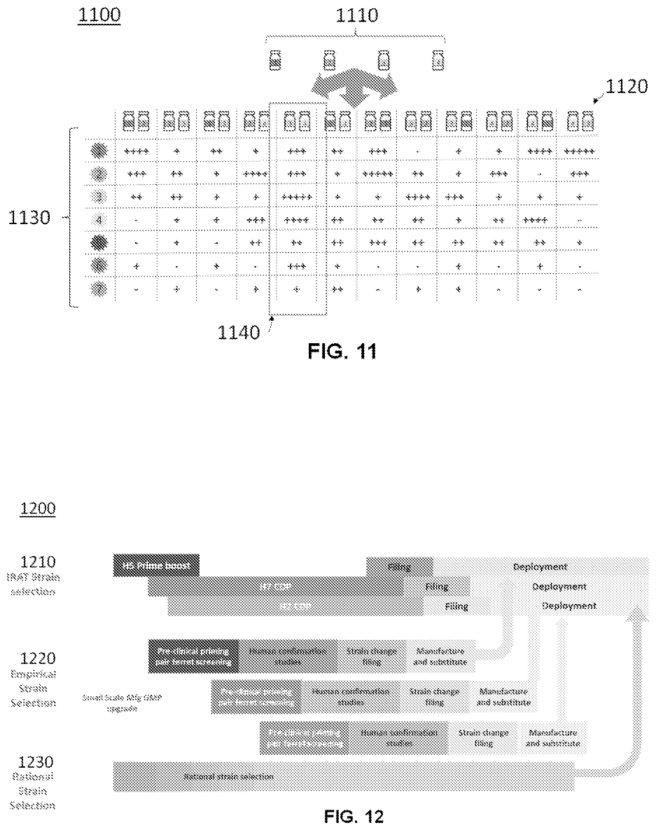

FIG. 12 depicts a flow chart of an exemplary vaccine deployment 1200 comprising an initial IRAT strain selection 1210 for rapid response followed by iterated empirical strain selection 1220 consistent with embodiments of the present disclosure. Optionally, additional methods of vaccine development such as, for example, bioinformatics-based rational antigen design, sa-mRNA antigen production, and/or antigenic cartography may proceed in parallel as depicted by 1230.

In some embodiments, the inventory of finished vaccines is selected with reference to an IRAT. In some embodiments, the inventory of finished vaccines is selected using antigenic cartography. In some embodiments, vaccines may be periodically added to and/or removed from the initial panel of vaccines. In some embodiments, the inventory of finished vaccines is periodically updated with vaccines selected from the initial panel of vaccines based on the clinical testing. In some embodiments, the method of empirically selecting the inventory of finished vaccines is iterated one or more times in parallel. In some embodiments, the method of empirically selecting the inventory of finished vaccines is non-synchronously iterated one or more times in parallel. In some embodiments, the method of empirically selecting the inventory of finished vaccines is iterated one or more times in series.

In the preceding disclosure, various example embodiments have been described with reference to the accompanying drawings. It will, however, be evident that various modifications and changes may be made thereto, and additional embodiments may be implemented, without departing from the broader scope of the disclosure as set forth in the claims that follow. The disclosure and drawings are accordingly to be regarded in an illustrative rather than restrictive sense.

Therefore, it is intended that the disclosed embodiments and examples be considered as examples only, with a true scope of the present disclosure being indicated by the following claims and their equivalents.

1. A method of selectively providing access to an inventory of pre-pandemic vaccines comprising:
identifying at least two Influenza Viruses of Pandemic Potential (IVPP) to provide available vaccines;
determining a stockpile requirement for vaccines against selected IVPPs among the at least two IVPPs;
obtaining finished vaccines against each of the selected IVPPs;
storing an inventory of the finished vaccines against each of the selected IVPPs;
preparing delivery logistics of the finished vaccines against at least one of the selected IVPPs in response to an access request; and
periodically obtaining further finished vaccines against one or more of the selected IVPPs and adding the further finished vaccines to the inventory of finished vaccines, such that the stored inventory of finished vaccines has a sufficient number of finished vaccines that have not reached their expiration date to meet the stockpile requirement.

2. The method of claim 1, wherein the manufacturing of finished vaccines includes vaccines against the previously selected IVPPs and additional IVPPs.

3. The method of claim 1, wherein finished vaccines are replaced in the inventory prior to the expiration date.

4. The method of claim 1, wherein the manufacturing schedule is correlated to the expiration date of the finished vaccines.

5. The method of claim 1, wherein the stockpile requirement is determined based on received information regarding one or more factors comprising a desired number of doses of vaccines and a number of selected IVPPs.

6. The method of claim 1, wherein the finished vaccines are delivered no more than two-weeks after the access request.

7. The method of claim 1, wherein an annual subscription fee is calculated based on the stockpile requirement.

8. The method of claim 1, wherein a vendor maintains the inventory of target vaccines.

9. The method of claim 1, wherein a manufacturer maintains the inventory of target vaccines.

10. The method of claim 1, wherein a customer only receives the finished vaccines after the finished vaccines are drawn down following the access request.

11. The method of claim 1, wherein the stockpile requirement is identified based on at risk groups comprising young children, patients, elderly patients, first responders and essential workers.

12. The method of claim 1, wherein the identified IVPPs are updated.

13. The method of claim 1, wherein the inventory of finished vaccines is updated to account for antigenic drift.

14. The method of claim 1, wherein the identified IVPPs include at least one IVPP chosen from H1, H2, H3, H5, H7, or H9.

15. The method of claim 1, wherein the identified IVPPs are identified with reference to an IRAT.

16. The method of claim 1, wherein the inventory of finished vaccines is stored as multi-dose vials, pre-filled syringes or a combination of both.

17. The method of claim 1, wherein the finished vaccines have a shelf life of at least 1 year.

18. The method of claim 1, wherein the further finished vaccines are manufactured in the time available between northern hemisphere vaccine production and southern hemisphere vaccine production.

19. The method of claim 1, wherein the IVPPs are identified at a date prior to a start of vaccine manufacturing, such that a duration between the date and the start of manufacturing meets or exceeds a preparatory phase duration.

20. The method of claim 1, wherein determining the stockpile requirement comprises:
producing an initial panel of vaccines wherein the panel comprises two or more different antigens;
screening combinations of two or more vaccines of the initial panel of vaccines in a non-human animal model;
selecting one or more of combinations for clinical testing in human patients; and
selecting one or more combinations for inclusion in the stockpile based on the clinical testing.
